(19) United States
(12) Patent Application Publication
(10) Pub. No.: US 2026/0027250 A1
(43) Pub. Date: Jan. 29, 2026

(54) STERILIZATION PACKAGING SYSTEM AND METHOD

(71) Applicant: Turbett Surgical, Inc., Henrietta, NY (US)

(72) Inventor: Robert E. Turbett, Penfield, NY (US)

(21) Appl. No.: 18/994,349

(22) PCT Filed: Jul. 24, 2023
(86) PCT No.: PCT/US2023/028464

§ 371 Date: Jan. 14, 2025

Related U.S. Application Data
(60) Provisional application No. 63/391,431, filed on Jul. 22, 2022.

(51) Int. Cl. A61L 2/26; A61B 46/10

(52) U.S. Cl. CPC A61L 2/26; A61B 46/10; A61L 2202/121; A61L 2202/18; A61L 2202/181; A61L 2202/24

(57) ABSTRACT

A sterilization packaging system for an object requiring sterilization in a sterilizer, the sterilization packaging system comprising a base having a support surface and a plurality of sides, the support surface configured to support an object requiring sterilization, a wrap sized to overlie the sides of the base and the object requiring sterilization, the wrap having a main body portion and a lower portion, a sealing interface between the lower portion of the wrap and the base, the sealing interface configured to form a continuous seal between the lower portion of the wrap and a first sealing surface, and a first drape moveable between an unfolded position and a folded position, wherein the first drape is sized to overlie at least a first portion of a support surface of the base in the folded position.

### BACKGROUND OF THE INVENTION

### Field of the Invention

The present invention relates to a sterilization packaging system and method for sterilizing surgical instruments and the like and more particularly to a sterilization packaging system comprising a base and a wrap and a method of using the same.

### Description of Related Art

Cleaning and sterilizing surgical instruments, laboratory supplies, and medical devices are important in many fields including, but not limited to, scientific research, veterinary medicine, and human medicine. Surgical instruments, for example, must be provided in a sterile condition to be able to be used in surgery. To reduce the risk of introducing harmful microorganisms to a patient or in scientific research, several steps are typically followed. First, the objects may be decontaminated and pre-cleaned. Next, the objects are cleaned, rinsed and dried. Following a thorough cleaning, the objects are sterilized. Sterilization is a term referring to any process that eliminates, removes, deactivates, or kills bacteria or other types of living microorganisms, including transmissible agents (such as fungi, bacteria, viruses, or spore forms) present on a surface, or contained in a fluid, or in medication, or in a compound such as biological culture media. Sterilization can be achieved by applying heat, steam, chemicals, such as using ethylene oxide (EO), irradiation, high pressure, and filtration or combinations thereof. In general, surgical instruments, laboratory supplies, and medical devices must be sterilized to a high sterility assurance level before use on a body. Examples of such instruments include scalpels, hypodermic needles, endoscopes, and implantable medical devices (IMD), such as artificial pacemakers.

A widely used method for heat sterilization is the autoclave, sometimes referred to as a converter. Autoclaves commonly use steam heated to 121-134° C. To achieve a degree of sterility, a holding time of at least 15 minutes at 121° C. at 100 kPA, or 3 minutes at 134° C. at 100 kPa is required. Additional sterilizing time is usually required for liquids and instruments packed in layers of cloth, as they may take longer to reach the required temperature.

One method of sterilization involves passing steam through a rigid container system, for example, a cabinet. For effective sterilization, steam needs to penetrate a cabinet load uniformly. Accordingly, the cabinet must not be overcrowded, and the lids of bottles and containers must be left ajar. During the initial heating of the chamber, residual air must be removed. Indicators should be placed in the most difficult places for the steam to reach to ensure that steam actually penetrates there.

A filter is typically placed over the vent to keep particles or extraneous materials from entering the cabinet before, during or after the sterilizing process. Once the sterilizing process is completed the filter needs to be removed and inspected by medical professionals to verify the integrity of the sterilizing process was maintained. If it is discovered during inspection that the filter did not remain intact, the sterilizing process has to be repeated with a new filter.

Another method of sterilization involves wrapping instruments, supplies, medical devices, or trays containing such objects, with a packaging material to allow sterilization of the contents of the tray, and to maintain the sterility of the tray contents until the wrap is opened. Traditionally, instruments that need to be reprocessed are provided in vented trays, allowing the sterilizing agent to flow through the tray, and sterilizing all the surfaces. In order to maintain sterility after removal from the sterilizer, the trays are wrapped in a material that allows the sterilizing agent to pass through, but protecting the inner tray from microorganisms until use. A multitude of packaging materials can be used for sterilization, including woven fabrics, which may be made of cotton, a cotton-polyester blend, or synthetic blend, or nonwoven materials, which may be made of plastic polymers, cellulose fibers, or washed paper pulp bonded under pressure into sheets. Wrapping instruments, supplies, medical devices, or trays containing such objects, in packaging material, including but not limited to wraps sometimes results in the packaging material tearing or puncturing, in which case the sterilization process must be repeated with new packaging material. Further, wrapping instruments in this manner provides a significant surface area to inspect after running the sterilization cycle of a sterilizer. Single instruments can be individually sterilized in a peel pouch. The pouch maintains the sterility of the instrument after the sterilization process is complete by protecting it from the contaminants in the environment. Peel pouches, however, are not efficient as multiple tools cannot be sterilized within the same peel pouch. Further, peel pouches are to be loaded into a sterilizer on its side to promote air removal and penetration of the sterilant as well as to discourage moisture retention. Peel pouches cannot include implants having several component parts. These component parts would need to be packaged in separate peel pouches before sterilizing, typically by gamma irradiation processing.

Alternatively, the trays may be placed in a rigid container utilizing filters that act as the wrap does, allowing the sterilizing agent in, but blocking microorganisms afterwards. The filter also allows the escape of the sterilizing agent or its byproducts. Once the sterilizing process is complete the filter needs to be removed and inspected by medical professionals to verify the integrity of the sterilizing process was maintained. If it is discovered during inspection that the filter did not remain intact, the sterilizing process has to be repeated with a new filter. Large containers have been made to handle multiple trays to allow a consolidation of steps, simplifying the process. These large containers are expensive to manufacture, however, and bulky to store. Further, the large containers hold several instruments, which collectively, is a significant weight load, requiring particular lifting devices to load and unload the container into a sterilizer and to place the container in storage.

After sterilization is complete, trays containing sterilized objects may be removed from the rigid container and placed on an operating room back table. Before such placement, the operating room back table is draped with a disposable sterile drape that covers the surface of the table to maintain a sterile field. The drapes are typically made of strong and absorbent material that is tear-resistant.

### BRIEF SUMMARY OF THE INVENTION

A first exemplary embodiment of the present disclosure provides a sterilization packaging system for an object requiring sterilization in a sterilizer, the sterilization packaging system comprising a base having a support surface and first, second, third and fourth sides, the support surface configured to support the object requiring sterilization and the base having a length and a width, a wrap sized to overlie the sides of the base and the object requiring sterilization, the wrap having a main body portion and a lower portion, a sealing device for sealing the lower portion of the wrap to each of the first, second, third and fourth sides of the base, a first drape having a length that is greater than the length of the base, wherein the first drape is configured to substantially encompass at least a first portion of the support surface of the base, and a second drape having a width that is greater than the width of the base, wherein the second drape is configured to substantially encompass at least a second portion of the support surface of the base.

A second exemplary embodiment of the present disclosure provides a method of sterilizing items in a sterilizer comprising the steps of deploying over a base having sides and a support surface for an item to be sterilized, a sterilization wrap material having a main body portion and a lower portion, the sterilization wrap material sized to overlie the base placing a sealing device around the sides of the base, the sealing device having a band including an inner portion, and outer portion and a compressible layer, the compressible layer circumscribing the inner portion of the band, and compressing the compressible layer against the lower portion of the wrap and the sides of the base to form a sealed volume enclosing the at least one item to be sterilized.

A third exemplary embodiment of the present disclosure provides a sterilization packaging system for an object requiring sterilization in a sterilizer, the sterilization packaging system comprising a base having a support surface and a dependent skirt having sides, the support surface configured to support the object, a wrap having a main body portion and a lower portion, the main body portion comprising a top panel and upper edge panels, wherein the upper edge panels comprise a rigid material configured to substantially hold a selected shape, and wherein the top panel comprises a transparent material providing at least one transparent wall for viewing the object, and wherein the lower portion comprises lower edge panels configured to overlap the sides of the dependent skirt, and a sealing device for sealing the lower portion of the wrap to the dependent skirt of the base.

A fourth exemplary embodiment of the present disclosure provides a sterilization packaging system for an object requiring sterilization in a sterilizer, the sterilization packaging system comprising a base having a support surface and a continuous sealing surface, the support surface configured to support the object requiring sterilization, a first drape sized to overlay at least a first portion of the object requiring sterilization, and a wrap sized to engage the continuous sealing surface to encompass the first drape and the object requiring sterilization.

A fifth exemplary embodiment of the present disclosure provides a method of preparing an object requiring sterilization in a sterilizer, the method comprising disposing the object requiring sterilization on a base, at least partially encompassing the object requiring sterilization within a first drape, enclosing the at least partially encompassed object requiring sterilization within the first drape within a wrap, and engaging the wrap with a continuous sealing surface of the base.

A sixth exemplary embodiment of the present disclosure provides a method of sterilizing items in a sterilizer comprising the steps of deploying over a base having a support surface for an item to be sterilized, a sterilization wrap material having a first sealing surface and a first expandable portion having a plurality of folds, the first expandable portion coupled to at least one of a first and second non-expandable portion, sealing the first sealing surface of the sterilization wrap material in the deployed configuration to form a sealed volume enclosing the at least one item to be sterilized, and placing a containment device over the sterilization wrap material, the containment device configured to reduce expansion of the sterilization wrap material during a sterilization cycle of the sterilizer, the deployed sterilization wrap material, base, and containment device forming a sterilization wrap assembly. In one exemplary embodiment, the step of deploying the sterilization wrap material further comprises moving a band circumscribing the base along support arms of the base to a first position, and, inserting a portion of the sterilization wrap material having the first sealing surface between the band and the support arms when the band is in the first position, and wherein the step of sealing the first sealing surface of the sterilization wrap material further comprises moving the band to a second position to secure the sterilization wrap material to the base, and locking the band in the second position. The step of deploying the sterilization wrap material may further comprise placing the base on top of the sterilization wrap material, and, wrapping the base having an item to be sterilized with the sterilization wrap material. The method may further comprise the steps of placing the sterilization wrap assembly into a sterilizer, running a sterilization cycle of the sterilizer, removing the sterilization wrap assembly from the sterilizer, removing the containment device from the sterilization wrap assembly, moving the band to the first position, confirming the integrity of the sterilization wrap material, and expanding the sterilization wrap material from a deployed configuration to an expanded configuration without separating the expandable portion from the first and second non-expandable portions to remove the sterilization wrap material from the base.

A seventh exemplary embodiment of the present disclosure provides a sterilization wrap material for sterilizing items in a sterilizer, the sterilization wrap material comprising a one-piece sheet member having a first portion of plurality of accordion folds, the plurality of accordion folds including a series of spaced and substantially parallel plates with alternating raised and recessed folds forming a corrugation pattern of accordion pleats traversing the sterilization wrap material (Area C), wherein the one-piece sheet is oversized to overlie a base having at least one item to be sterilized. The sterilization wrap material may further comprise a first and second longitudinal fold line and first and second lateral fold line forming a first and second top panel, a first side panel formed by the first longitudinal fold lines and the second lateral fold line, a second side panel formed by the second longitude fold line and the second lateral fold lines, a third side panel formed by the first longitudinal fold line and the first lateral fold line, and a fourth side panel formed by the second longitudinal fold line and the first lateral fold line. Additionally, the plurality of accordion folds may form an expandable configuration, and wherein the plurality of accordion folds are moved from a contracted state to an expanded state when the sterilization wrap material is removed from the base. In one configuration, the first portion, first and second top panels, and first, second, third and fourth side panels are unseparable.

An eight exemplary embodiment of the present disclosure provides a method of using the sterilization wrap material described in the preceding paragraph, the method comprising folding the a one-piece sheet member along the first and second longitudinal fold lines and the first and second lateral fold lines to form folds along each of the first and second longitudinal fold lines and the first and second lateral fold line, respectively, and reinforcing each of the folds to form a pre-shaped bag.

A ninth exemplary embodiment of the present disclosure provides a preformed sterilization barrier system for at least one item to be sterilized in a sterilizer comprising a sterilization wrap material having a first sealing surface and a first expandable portion having a plurality of folds, wherein in a deployed configuration the sterilization wrap material overlies a base having a support surface, a bottom surface, and a dependent skirt, and wherein in an expanded configuration, at least one of the plurality of folds is expanded to increase the size of the sterilization wrap material, and, a first seal for maintaining a sealed interface between the first sealing surface of the sterilization wrap material and the base in the deployed configuration to form a sealed volume enclosing the at least one item to be sterilized. The sterilization wrap material of the preformed sterilization barrier system may further comprises a first non-expandable portion adjacent the first expandable portion, wherein in the expanded configuration the sterilization wrap material is expanded without separating the first expandable portion from the non-expandable portion. In a configuration, the first seal is formed around the entire base between the first sealing surface of the sterilization wrap and the dependent skirt. In a configuration, the first seal is formed around the entire base between the first sealing surface of the sterilization wrap and the bottom of the base. In one configuration, the plurality of folds expand the material in a lateral direction in the expanded configuration. A sealing device, in one configuration, is configured to form the first seal between the first sealing surface of the sterilization wrap material and the base in the deployed configuration. In a configuration, the sealing device is a clamp and in another configuration, the sealing device is a tension band. The base can include a channel extending circumferentially around the dependent skirt of the base, wherein the channel is sized to receive the sealing device. The preformed sterilization barrier system in a configuration further comprises a containment device configured to reduce expansion of the sterilization wrap material during a sterilization cycle of the sterilizer. The containment device can include a frame overlying the sterilization wrap material in the deployed configuration. In one configuration, the containment device is a strap positioned over the sterilization wrap material when the sterilization wrap material is in the deployed configuration. The plurality of folds, in one configuration, are accordion folds having a series of spaced and substantially parallel plates with alternating raised and recessed folds forming a corrugation pattern of accordion pleats longitudinally traversing the sterilization wrap material. In one configuration, the first expandable portion is adjacent a second non-expandable portion opposite the first non-expandable portion, wherein in a collapsed configuration, the plurality of folds and the first non-expandable portion overlie the second non-expandable portion. In the deployed configuration, the plurality of folds may be at least partially contracted. In one configuration, the plurality of folds are accordion folds moveable to expand the volume of the sterilization wrap material in the expanded configuration without separating the first and second non-expandable portions from the expandable portion. The preformed sterilization barrier system, in one configuration, further comprising a releasable adhesive to maintain at least a portion of the expandable portion of the sterilization wrap material in a contracted position when the sterilization wrap material is in the deployed configuration. The base may comprise at least one tray. In another configuration, the base is a surgical table. The sealing device, in one configuration, is a band configured to seal the sterilization wrap material to the base, wherein the base further includes a plurality of arms projecting from the base, and wherein the arms are configured to support the band. In one configuration, the band is moveable along the plurality of arms in a first direction for installing the sterilization wrap material and in a second direction for securing the sterilization warp material in the deployed configuration, wherein the sterilization wrap material is between each of the plurality of arms and the band in the deployed configuration. The band can include a releasable lock.

A tenth exemplary embodiment of the present disclosure provides a sterilization packaging system for an object requiring sterilization in a sterilizer, the sterilization packaging system comprising a base having a support surface and a dependent skirt having sides, the support surface configured to support the object, a wrap having a main body portion and a lower portion, the main body portion comprising a top panel and upper edge panels, wherein the upper edge panels comprise a rigid material configured to substantially hold a selected shape, and wherein the top panel comprises a transparent material providing at least one transparent wall for viewing the object, and wherein the lower portion comprises lower edge panels configured to overlap the sides of the dependent skirt, and, a sealing device for sealing the lower portion of the wrap to the dependent skirt of the base. In one configuration, the top panel includes an inner perimeter of the rigid material and an opening, and the transparent material overlies the inner perimeter. The transparent material in one configuration is adhesively bonded to the inner perimeter of the top panel. In another configuration, the transparent material is heat sealed to the inner perimeter of the top panel. In one configuration, the rigid material is two layers of filter paper material. In another configuration, the rigid material is moldable filter paper configured to hold a shape selected to conform to the shape of the base. The sterilization packaging system may further comprise a first drape having a length that is greater than a length of the base, wherein the first drape is configured to substantially encompass at least a first portion of the support surface of the base. The first drape may further comprise a first end, a second end and a middle portion connecting the first and second ends, wherein the first end of the first drape extends around one of the sides of the base and over at least a first portion of the support surface, and wherein the second end of the first drape extends around another side of the base and over at least a second portion of the support surface. The sterilization packaging system may further comprise a second drape having a width that is greater than a width of the base, wherein the second drape is configured to substantially encompass at least a second portion of the support surface of the base. The second drape may further comprise a first end, a second end and a middle portion connecting the first and second ends, wherein the first end of the second drape extends around one of the sides of the base and over at least a third portion of the support surface, and wherein the second end of the second drape extends around another one of the sides of the base and over at least a fourth portion of the support surface.

An eleventh exemplary embodiment of the present disclosure provides a method of sterilizing items in a sterilizer comprising the steps of deploying over a base having a plurality of sides and a support surface for an item to be sterilized, a sterilization wrap material having a main body portion and a lower portion, the sterilization wrap material sized to overlie the base, placing a sealing device around the sides of the base, the sealing device having a band including an inner portion, and outer portion and a compressible layer, the compressible layer circumscribing the inner portion of the band, compressing the compressible layer against the lower portion of the wrap and the sides of the base to form a sealed volume enclosing the at least one item to be sterilized, deploying a first drape having a first end, a second end and a middle portion connecting the first and second ends, wherein the first end of the drape extends around one of the sides of the base and wherein either the middle portion or the first end portion extends over at least a first portion of the support surface of the base, and deploying a second drape having a first end, a second end and a middle portion connecting the first and second ends, wherein the first end of the drape extends around one of the sides of the base and wherein either the middle portion or the first end portion extends over at least a second portion of the support surface of the base. The method, in one configuration, further comprises the steps of engaging a compression latch to compress the compressible layer against the lower portion of the wrap and the sides of the base to form a seal, the compression latch having a tamper-evident device to indicate whether the compression latch been released after engaging. In a configuration, the method further comprising the steps of sterilizing a plurality of bases, each having a sealed volume enclosing the at least one item to be sterilized, and placing one of the plurality of bases having been sterilized in a sterilizer on one of a plurality of shelves of a rack, each of the plurality of shelves configured to hold one of the plurality of bases.

A twelfth exemplary embodiment of the present disclosure provides a sterilization packaging system for an object requiring sterilization in a sterilizer, the sterilization packaging system comprising a base having a support surface and a plurality of sides, the support surface configured to support an object requiring sterilization, a wrap sized to overlie the sides of the base and the object requiring sterilization, the wrap having a main body portion and a lower portion, a sealing interface between the lower portion of the wrap and the base, the sealing interface configured to form a continuous seal between the lower portion of the wrap and a first sealing surface, and a first drape moveable between an unfolded position and a folded position, wherein the first drape is sized to overlie at least a first portion of a support surface of the base in the folded position. In one configuration, the sterilization packaging system further comprises a second drape moveable between an unfolded position and a folded position, wherein the first drape is sized to overlie at least a second portion of a support surface of the base in the folded position. In one configuration, the lower portion includes a tearaway portion comprising a tear line and a tear tab, and the main body portion is detachably connected to the lower portion along the tear line. In one configuration, the wrap further comprises a top panel and a plurality of side panels extending therefore, the top panel including an inner perimeter of the rigid material and an opening, and wherein the transparent material overlies the inner perimeter. In one configuration, the transparent material is adhesively bonded to the inner perimeter of the top panel. In another configuration, the transparent material is heat sealed to the inner perimeter of the top panel. In yet another configuration, the wrap further comprises corners, each corner having at least one inner pleat and a panel, the panel configured to be positioned proximate a side of the wrap to provide continuous wrap material at the corner.

### BRIEF DESCRIPTION OF THE SEVERAL VIEWS OF THE DRAWING(S)

FIG. 1 is an exploded view of an exemplary embodiment of the present disclosure sterilization packaging system.

FIG. 2 is a perspective view of the exemplary embodiment of the present disclosure sterilization packaging system shown in FIG. 1 in an assembled position, wherein the security device includes a latch.

FIG. 3 is a perspective view of a securement device of the present sterilization packaging system.

FIG. 4A is a top view of an exemplary embodiment of a sterilization wrap of the present disclosure sterilization packing system.

FIG. 4B is a perspective view of the sterilization wrap of FIG. 4A showing the wrap in a partly assembled position.

FIG. 5A is a top view of another exemplary embodiment of a sterilization wrap of the present disclosure sterilization packing system.

FIG. 5B is a perspective view of the sterilization wrap of FIG. 5A showing the wrap in an assembled position.

FIG. 6 is a top perspective view of a sterilization wrap having a transparent window.

FIG. 7 is a perspective view of another exemplary embodiment of the present disclosure sterilization packaging system.

FIG. 8 is an exploded view of another exemplary embodiment of the present disclosure showing (i) a first and second drape to be disposed around a tray, and, (ii) the sterilization wrap, each of the present disclosure sterilization packaging system.

FIG. 9 is an exploded view of the exemplary embodiment of FIG. 8 showing the first and second drape disposed around the tray.

FIG. 10 is an exploded view of another exemplary embodiment of the present disclosure showing a first and second drape to be disposed around a base and the sterilization wrap of the present disclosure sterilization packaging system.

FIG. 11 is perspective view of an exemplary embodiment of a holding rack of the present disclosure sterilization packaging system.

FIG. 12 is a perspective view of an exemplary embodiment of a transport table of the present disclosure sterilization packaging system.

FIG. 13 is a flow diagram in accordance with a method and apparatus for performing exemplary embodiments of this disclosure.

FIG. 14 is a flow diagram in accordance with a method and apparatus for performing exemplary embodiments of this disclosure.

FIG. 15 is a top view of an exemplary embodiment of a sterilization wrap of the present disclosure sterilization packing system.

FIG. 16 is a perspective view of the sterilization wrap shown in FIG. 15 in a deployed position.

### DETAILED DESCRIPTION OF THE INVENTION

It should be appreciated that the same reference numbers appearing in different figures identify the same structural elements of the present invention. While the description of the present invention includes what is currently considered to be the preferred configurations, it should be appreciated that the present invention is not limited to such configurations. Moreover, it should be appreciated that the present invention is not limited to the particular methodology, materials and modifications described herein and that the terminology used herein is not intended to limit the scope of the present invention. The scope of the present invention is therefore to be determined solely by the appended claims.

The invention pertains to a sterilizing wrap system to provide significant vent to volume ratios for purposes of sterilizing instruments, medical devices, and the like during sterilization in a sterilizer. The term sterilizer includes, but is not limited to, a housing or device defining an interior retaining the sterilization system and in which a controlled environment is created to impart a desired sterilization. Sterilizers include autoclaves; hot air ovens; ethylene oxide; low temperature steam and formaldehyde; sporicidal chemicals; irradiation; chlorine dioxide (CD) gas sterilization; hydrogen peroxide; vaporized hydrogen peroxide; hydrogen peroxide plasma; electron beam and gas plasma devices.

The system provides the benefits of both wrapping techniques and rigid container systems. By utilizing a base such as a table, tray or a plurality of trays as a rigid component without a door, one or more instruments may be placed on it. By utilizing a wrap as a protective cover, significant vent to volume effects can be realized. This results in a shorter time required for the sterilizing agent or its byproducts to be vented. Further, when a base, such as an operating room back table or tray or trays, is used, there is typically no need to transfer the instruments from a rigid container onto a back table for use in the operating room, as the back table is integral to the sterilizing wrap system and thus, the instruments are already organized on the table and presentable after sterilization. Moreover, by including a first drape and, in some configurations, a second drape, as part of the system, additional or separate draping of the operating room back table is not needed. By “operating room back table” or “back table” it is meant to generally refer to a table used in the operating room for holding surgical instruments. The operating room back table is sometimes known as an instrument table or a work table. The back table can be made of stainless steel, plastic, or other rigid materials, and can be any shape or size. Typically, the back table is a rectangular shaped table having legs and/or a pedestal which may or may not be adjustable and/or removable.

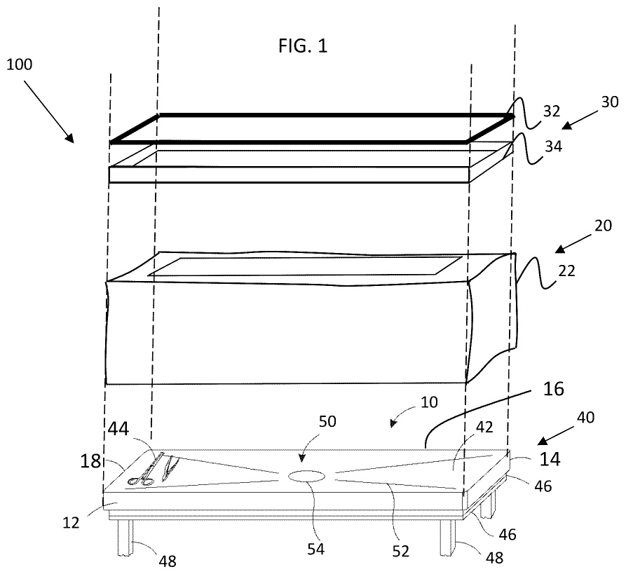

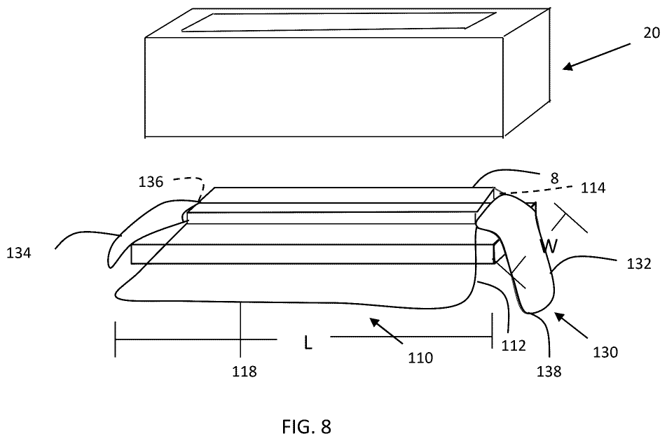

Generally, the sterilization packaging system 100 includes a base 10 providing a support surface 42 and a wrap 20 providing a protective cover. The base 10 may be any shape and size for providing a support surface. For example, although base 10 is shown in a rectangular configuration, the base 10 may be any shape, including, but not limited to, circular, oval, square, and elliptical. The base 10 in this configuration is rigid and may be a tray, table, sheet, plate, or any other type of rigid, supportive base. For example, in one configuration, the base 10 may be a tray 8 as shown in FIG. 8. In one exemplary embodiment, the base 10 includes sidewalls 12, 14, 16, 18. It should be appreciated that the sterilization wrap 20 may take many different shapes and forms, including but not limited to a sheet, bag, pouch, and a partially formed bag. Referring now to the figures, FIG. 1 is an exploded view of the sterilization packaging system 100 having a base 10, a wrap 20, and a securement device 30. In this configuration, the securement device 30 includes a compressible layer 32. As shown in FIG. 1, the base 10 in one configuration is an operation room back table 40 having a support surface 42 for supporting at least one object 44 requiring sterilization by a sterilizer. Objects 44 requiring sterilization may include, but are not limited to surgical tools and instruments, implants, laboratory supplies, and medical devices. In one configuration, the base 10 includes a dependent skirt 46 and may be detachable from legs 48 or a pedestal (not shown). The base 10 may also include a flange or sidewalls (not shown) along the perimeter edge of the support surface 42 for retaining the objects 44 on the support surface 42, especially during transportation when objects may otherwise shift. Alternatively, or additionally, the base 10 may include dividers, shelves, or sectioned portions for maintaining objects 44, including but not limited to, surgical instruments and/or trays, on particular sections of the base 10. The base 10 may further include additional fasteners, including but not limited to Velcro, magnets, and the like to releaseably hold the objects 44 in place. The base 10 may include a laser outline of at least some objects 44 identifying a preferred location for particular objects. Other features for organizing and maintaining objects 44 on the base 10 are intended to be included within the scope of the invention as claimed.

In some configurations, the base 10 may include a drain system 50 for draining sterilizing agent and/or liquid condensate formed during the sterilization process. The base 10 may include, for example, a sloped surface 52, for example, as shown in FIG. 1, to direct drainage to a predetermined area. The sloped surface 52 may be of any slope and may be a straight line (angular) or curved. In one configuration, the base 10 includes at least one drainage well having a downwardly sloping surface 52 arrange to direct condensate to a drain aperture 54. In one configuration, the drain aperture 54 is approximately 0.5 to 5 inches in diameter. In another configuration, the drain aperture 54 is approximately 2-3 inches in diameter. The drain aperture 54 may be any shape and size that allows liquid condensate or liquid sterilant to drain from the surface of the base 10. The drain aperture may further include a drain check valve and/or at least one filter. The surface 52 may be frusto-conical shaped to further direct sterilizing agent and condensate to the drain aperture 54. Alternatively, the drain system 50 may include a continuous decline directing sterilizing agent and/or liquid condensate to a drain well or aperture 54 located proximate one side of the base 10. It should be appreciated that the drain well or aperture 54 or a plurality of drain wells and/or apertures 54 may be positioned anywhere on the floor of the base, with the floor configured to permit drainage thereto. In yet another configuration, the drain system 50 may include a sloped surface or decline without a drain well or aperture 54, which directs sterilizing agent and/or liquid condensate to an edge of the floor of the base 10. The drain system 50 many include a filter or filters within the aperture. It should be appreciated by those having ordinary skill in the art that other drainage systems that allow for sterilizing agent and/or liquid condensate to drain from the floor of the base 10 are possible and these modifications are intended to be included within the scope of the claims. The base 10 may further include a holder to maintain the objects 44 above the drain system 50. The holder may be, for example, a plate having perforations, apertures, or wells, or a tray configured to be suspended above the base 10, among other things. The holder may be integral to the base 10 or removeable from the base 10. In one exemplary embodiment, the holder is tray 8 as shown in FIG. 8. It should be appreciated that there are many configurations known in the art to hold instruments, and in certain embodiments hold the instruments above a drain system, and these configurations are intended to be included within the spirit and scope of the claims.

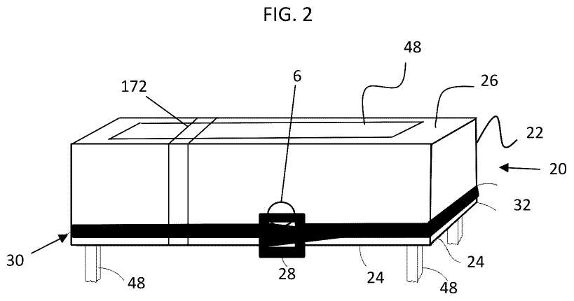

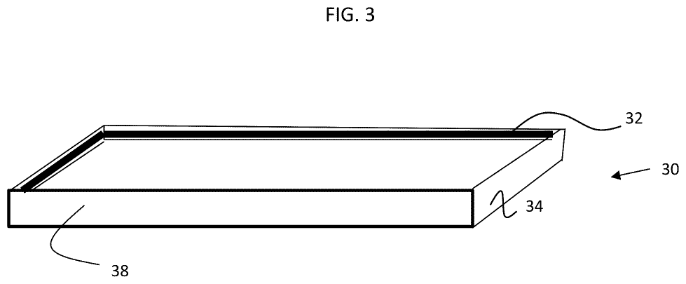

As shown in FIGS. 1-3, in an exemplary embodiment the sterilization packaging system 100 includes a securement device 30. The securement device 30 in one configuration is a band 34 having an inner portion 36, an outer portion 38, and a compressible layer 32. In one configuration, the band 34 is a metal band having sufficient transverse flexibly for conforming to the length and width of the sidewalls of the base 10. In another configuration, the band 34 is an elastic band having enough elasticity to stretch over the base 10 while also having enough tension to compress the compressible layer 32 against the wrap 20, and the wrap 20 against a sealing surface of the base 10. The compressible layer 32 may be permanently affixed to the inner portion 36 of the band 34 or removably secured to the inner portion 36 of the band 34. In one configuration, the compressible layer 32 is a closed cell foam and may further include a flat sealing surface for engaging the sealing surface with the sterilization wrap 20 in between. In one configuration, the securement device 30 includes a compression latch 28, as shown in FIG. 2, which is moveable between an open position and a closed position. The compression latch 28 may include a tamper-evident device 6 capable of indicating whether the compression latch 28 has been moved from the closed to the open position. In the open position, the securement device is disposed over the sterilization wrap 20 to secure the sterilization wrap 20 to the base 10. Then, the compression latch 28 is engaged to compresses the securement device 30 and the compressible layer 32 against the sidewalls of the base 10, for example, sidewalls 12, 14, 15, and 18, to provide a sealed interface between the compressible layer 32 and the sterilization wrap 20. Thus, the base 10 includes a support surface 42 and a perimetric sealing surface. The securement device 30, in one configuration, further provides a sealed interface between the securement device 30 and the sterilization wrap 20 and also between the base 10 and the sterilizing wrap 20. That is, the securement device 30 exerts a radially directed force on an inner surface of the base 10 to compress the compressible layer 32 and sterilization wrap 20 into sealing engagement with the base 10. It should be appreciated that the sealed interface between the base 10 and the sterilization wrap 20 may be achieved by force of the securement device 30 against the base 10. Thus, a seal is formed along the base 10 between the wrap 20 and a sealing surface without requiring adhesives—that is, without requiring glue, sealant polymer, tape or similar materials. In another exemplary embodiment, the sealed interface is formed by any type of mechanical or non-mechanical seal, including but not limited to a clamp, or an applied sealant, including but not limited to an adhesive, sealant polymer, or tape. Further, it should be appreciated that other types of securement devices 30 that maintain a sealing interface between the wrap 20 and the base 10 are contemplated and these modifications are intended to be included herein. For example, shrink wrap or Krayton material may be used to form the clamp around the base 10 such that a sealed interface is maintained between the wrap 20 and the base 10. The wrap 20, in one configuration includes multiple seals. For example, the wrap 20 may include two or three sealing interfaces between the wrap 20 and the base 10. In one configuration, for example, the wrap 20 includes two continuous adhesive seals. The sterilization packaging system 100 may be sized to accommodate an operating room back table. In one example embodiment, the sterilization packaging system is approximately 24 inches in length, 22.5 inches in width, and 12 inches in height. However, the size of the sterilization packaging system can vary. In certain embodiments, the length of the sterilization packaging system can be in the range of 12 inches to 36 inches. As shown in FIG. 2, the sterilization packaging system may include a non-expansion device to contain the wrap during a sterilization cycle of the sterilizer. The non-expansion device in one configuration is a strap 172 that secures to at least two of the sides of the base 10.

In one configuration, the sealing surface is the sidewalls 12, 14, 16, and 18 of the base 10 extending from a perimeter edge of the support surface 42. In another configuration, the sealing surface is the dependent skirt 48 of the base 10. In yet another configuration, the sealing surface is an upper outer wall (not shown) extending above the perimeter edge the support surface 42. For example, in one configuration, the base 10 is a tray 8 and the sealing surface is an upper outer wall extending above the perimeter edge of the support surface 42. Typically, base 10 is comprised of stainless steel, and thus the sealing surface is typically stainless steel. It should be appreciated, however, that base 10 may be comprised of other types of materials that can withstand exposure to a sterilizing agent without degradation, and thus the sealing surface is not limited to such material. The sealing surface may include a channel (not shown) at least partially circumscribing the sealing surface. Further, more than one sealing surface may be used, creating two or more sealing interfaces.

The sterilization wrap 20 may comprise many various shapes and styles and may be applied over the support surface 42 of the base 10 or from under the support surface 42 of the base 10. Typically, the sterilization wrap 20 is a rigid material configured to substantially hold a selected shape. In one configuration, the wrap 20 is comprised of a material that is resistant to tears. The sterilization wrap 20 may be a single layer or multi-layer material. The wrap 20 may comprise layers of different material. The material may be made with spunbound, meltblow, spunbound laminate (SMS) fabric, polypropylene, Tyvek, or any other type of material that provides a filtration capability. The materials may alternatively be one that can withstand gamma radiation. For example, the material may comprise a material that is gas-impermeable and which provides a strong and durable sterile barrier. In yet another configuration, the material is gas and water vapor permeable but not permeable by liquid. In this configuration, the material allows air and steam to pass through, but not by liquid droplets.

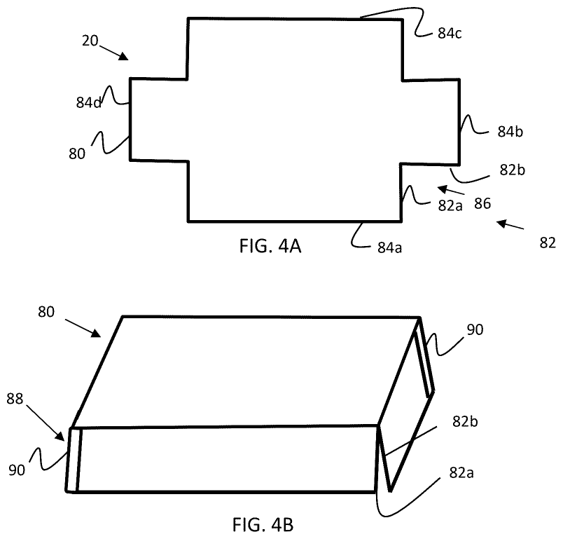

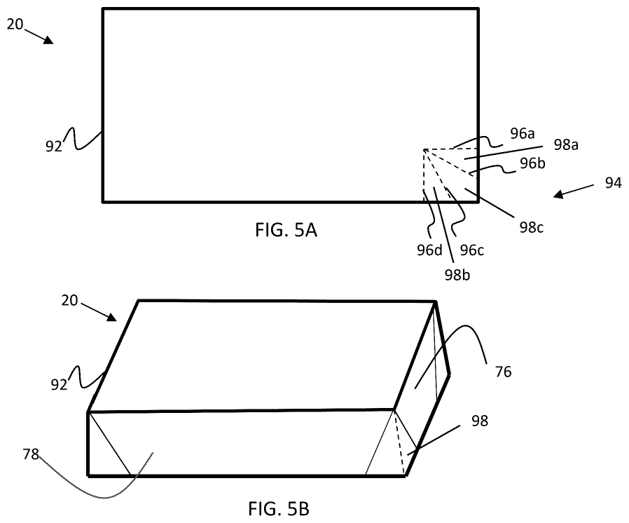

The wrap 20 material is selected based on the desired filtering and the type of sterilization process used. For example, a Tyvek and plastic combination would not be used in a steam sterilization process as it would melt. However, Tyvek and plastic combinations can be used in ethylene oxide (EtO) sterilizers. A filtering material can be used when steam must penetrate the material. In some configurations, the sterilization wrap 20 is a single-use material, and in other configurations, the sterilization wrap 20 is reusable. Further, the sterilization wrap 20 may include a single type of material or be constructed of multiple materials. Turning to FIGS. 4A and 4B, the wrap 20 may be formed of a filter paper blank 80 wherein each corner 82 has a cut-away portion 86 to form panels 84a, 84b, 84c, 84d. The end of each panel 84a, 84b, 84c, 84d forms a sealing end, for example sealing ends 82a, 82b, which can be sealed together to form a closed end 88. For example, sealing end 82a and corresponding sealing end 82b may be coupled by ultrasonic welding, adhesive, tape, or by other manufacturing methods known in the art. As shown in FIGS. 4A and 4B, in one configuration, sealing ends 82a, 82b are sealed together with adhesive tape 90.

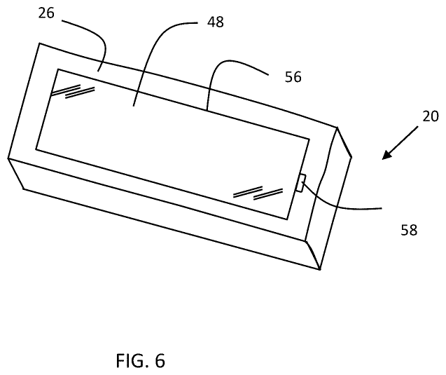

As shown in FIGS. 5A and 5B, the wrap 20 in another configuration is formed of a filter paper blank 92, wherein each corner 94 is folded to allow for a continuous material around each corner 94. In one configuration, each corner 94 of the wrap 20 includes fold lines 96a, 96b, 96c, and 96d. Fold lines 96a, and 96b form an inner pleat 98a on one of the sides 76 of the wrap 20, while fold lines 96c and 96d form an inner pleat 98b on an adjacent side 78 of the wrap. Fold lines 96b and 96c form panel 98c that folds over the outer portion of the wrap 20 on both of the adjacent sides 76, 78. The other corners 94 of the blank 92 can be folded in a similar manner thereby providing a continuous material around the perimeter of the base 10. The securement device 30 can then be disposed over the wrap 20 to secure the wrap 20 to the base 10. In one exemplary embodiment, the wrap is oversized to allow some expansion during the sterilization process. In another exemplary embodiment, the corners 94 are held together by ultrasonic welding, adhesive, tape, or by other manufacturing methods known in the art.

As shown in FIG. 2, wrap 20 may include a main body portion 22 and a lower portion 24. As shown in FIGS. 2 and 6, in one configuration, the main body portion 22 includes a top panel 26 having a transparent window 48 for permitting a user to view the contents within the sterilization packaging system 100. The transparent window 48 in one configuration comprises a clear plastic film. Although the clear plastic film is sealed to the wrap 20 material before and during use, the clear plastic film may be removed by the user to allow the user to access the contents within the wrap 20 as shown in FIG. 6. For example, the wrap 20 may include an opening in the top panel 26 formed by an upper edge portion 56 in the top panel 26. The opening is occluded by the transparent window 48. For example, in one configuration the perimeter edge of the transparent window 48 overlaps the upper edge portion 56 of the wrap 20 and is removably bonded to the wrap material with an adhesive, heat seal or the like, such that at least a portion of the transparent window 48 is separable from the wrap materials 20 when force is applied by a use. The transparent window 48 may include a tab 58 to allow the user to easily pull and separate the transparent window 48 or a portion thereof from the upper edge portion 56.

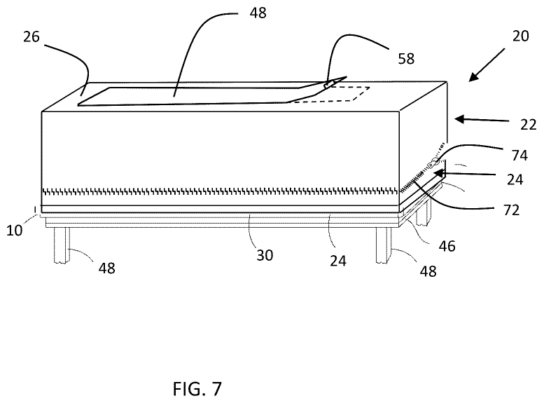

As shown in FIG. 7, in certain configurations, the sterilization packaging system 20 includes a frangible line 72 circumscribing the sterilization wrap 20 between the main body potion 22 and lower portion 24. The frangible line 72 may include, but is not limited to, frangible portions, perforations, cutting guide lines, scores, embossments, seams, or combinations thereof. The frangible line 72 enables the main body portion 22 to be detachable from the lower portion 24 along the frangible line 72. A tear tab or zipper 74 may be included along the frangible line 72 to facilitate the detachment of the main body portion 22 to the lower portion 24. The frangible line 72 in one configuration comprises a tear line along the perimeter of the lower portion 24 of the wrap 20. The frangible line 72 may include an additional, tear resistant material coupled to the frangible line 72. In one configuration, the securement device 30 holds the lower portion 24 while the main body portion 22 is detached. Engaging the tear tab or zipper 74 separates the main body portion 22 and the lower portion 24. Since the lower portion 24 is sealed to the base 10, the main body portion 22 can be removed while the lower portion 24 drapes down to serve as protective drape when the sterilization packaging system 20 is in a sterile field.

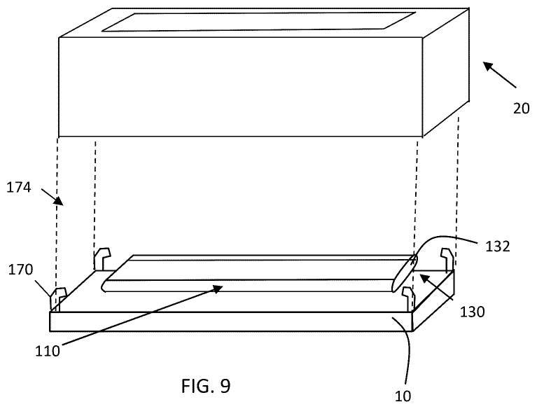

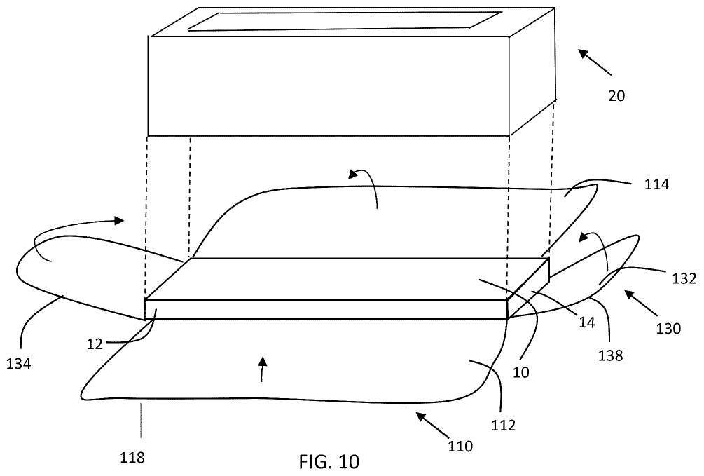

Further, as shown in FIGS. 8, 9 and 10, the base 10, or at least one tray 8, can be draped before deploying the sterilization wrap 20 to reduce the risk of contamination when the wrap 20 is removed after the sterilization process is complete, that is, after the sterilization packaging system 100 has completed a sterilization cycle in a sterilizer, such as an autoclave. In one configuration, as shown in FIG. 8, a drape 110 includes a first end 112, an oppositely arranged second end 114 so as to form two ends of the drape 110, and a middle portion between the first and second ends (the middle portion located underneath the tray 8 in FIG. 8 and underneath base 10 in FIG. 10). The drape 110 further includes a first side 118 which is substantially parallel to the side 12 (see FIG. 1) of the base 10 or the tray 8 having a length. In one configuration, the first side 118 of the drape 110 has a length that is at least twice the length of the tray 8 and the first end 112 and second end 114 have a width that is substantially the same size as the width of the tray 8. Typically the drape 110 has a length that is between two and four times the length of the base 10 or tray 8. In one configuration, the first and second ends 112, 114 are configured to be folded around each of two sides 12, 16 of the base 10 or corresponding sides of the tray 8 to protect contents on that portion of the base 10 or tray 8 from becoming contaminated when the sterilization wrap 20 is removed. In one configuration, the sterilization packaging system 100 further includes a drape 130 having a first end 132, an oppositely arranged second end 134 so as to form the two ends of the second drape, and a middle portion 136 between the first and second ends 132, 134. The drape 130 further includes a first side 138 between the first end 132 and the second end 134, the first end 132 and second end 134 having a length that is substantially the same size as the length of the base 10 or the tray 8, and the first side 138 of the drape 130 being substantially parallel to the width of the base 10 or the tray 8. The width in one configuration is between two and four times the width of the base 10 or tray 8, wherein each of the first and second ends 132, 134 is configured to be folded over the base 10 or tray 8. In a configuration, the first and/or second drapes 110, 130 enable the sterilized objects contained on the base 10 or the tray 8 to be used directly, that is, without transferring the sterilized objects 44 from the base 10 or tray 8 to a separately draped table. In one configuration, upon removal of the sterilization wrap 20, the first and/or second drapes 110, 130 are unfolded from the base 10 or tray 8 to drape below the surface of the base 10 or tray 8. The first and/or second drapes 110, 130, in one configuration, move from a folded position for example, as shown in FIG. 9, to an unfolded position, for example as shown in FIG. 8, when the sterilization wrap 20 is removed. In another configuration, the first and/or second drapes 110, 130 are moved by an operator from the folded position to an unfolded position. Thus, in the unfolded position, the first and second drapes 110, 130, can serve as drapes that maintain a sterile field in an operating room or other area where a sterile field is required. It should be appreciated that the drape 110 and drape 130 are not intended to be limited in shape as size. Generally, the drape or drapes is/are oversized from the size of the applicable base 10 or tray 8 and the ends of the drape or drapes overlap with each other over the top of the applicable base 10 or tray 8. In one configuration, the drape 110 overlies a portion of the support surface 42 of the base 10 or a portion of the tray 8. In one configuration, the drape 130 overlies a portion of the support surface 42 of the base 10 or a portion of the tray 8. In another configuration one or both of the first and second drapes 110, 130 overlie substantially the entire support surface 42 of the base or of the tray 8. In yet another configuration, one or both of the first and second drapes 110, 130 overlie a portion of an object 44 to be sterilized. In a further configuration, one or both of the first and second drapes 110, 130 overlie the entire, or substantially the entire object 44 to be sterilized. It should be appreciated that the sterilization packaging system shown in FIGS. 8, 9, and 10 may include the additional features described herein and shown in FIGS. 1, 2, 3, 4A, 4B, 5A, 5B, 6, and 7. By way of non-limiting example, the sterilization packaging system shown in FIGS. 8, 9, and 10 may include the securement device 30 described in FIGS. 1-3. As shown in FIG. 9, wrap supports 170 extend from the base 10 in one configuration The wrap supports 170 in one configuration are collapsible. In another configuration, wrap supports 170 are removeable.

The wrap 20 and the support surface 42 of the base 10 when sealed defines an interior area 174 and provides a continuous barrier to bacteria or other types of living microorganisms, including transmissible agents (such as fungi, bacteria, viruses, or spore forms). The drape 110 and, in some configurations, the drape 130 may form a secondary continuous barrier within the interior area and may include a secondary seal within the interior area 174. In one configuration, the drape 110 and drape 130 are sealed with adhesive, tape or other sealing material to form the secondary seal. In another configuration, only one drape 110 or 130 is used to provide a secondary continuous barrier, which in some configurations includes a secondary seal. It should be appreciated that drape 110 and/or drape 130 may be sized according to whether one drape or two drapes are desired to provide the continuous barrier and optionally the secondary seal.

The sterilization wrap 20 can define a venting pass through area 110 of the sterilization packaging system 100. The venting pass through area 110 refers to the total available area for the ingress and egress of a gas, vapor, and/or liquid to and from the interior volume of the sterilization packaging system 100. By “venting pass through area to volume ratio,” it is meant the ratio of the total venting pass through area of a sterilization packaging system 100 to the interior volume ratio of the sterilizing wrap system. This venting pass through area to volume ratio increases the area exposed to the sterilizing agent and reduces the amount of dry time required to provide no visible condensate, moisture, or sterilizing agent on the sterilized objects.

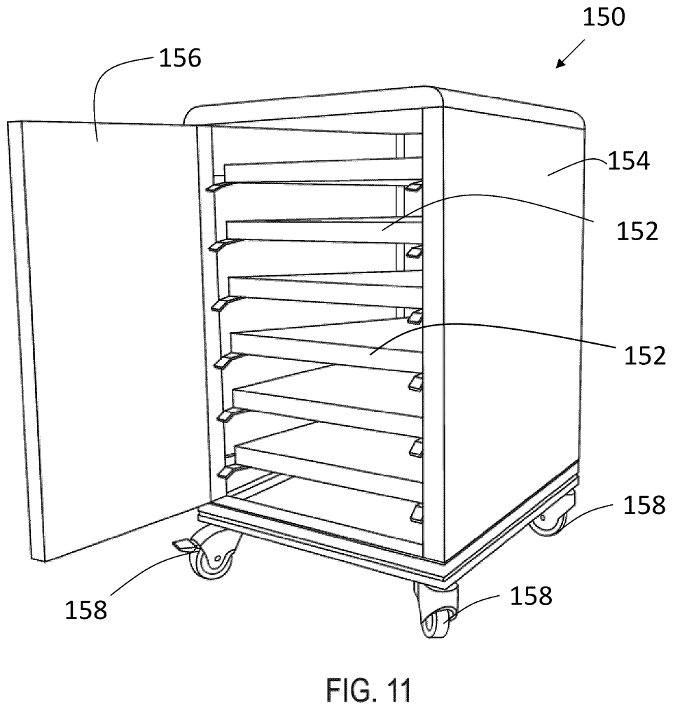

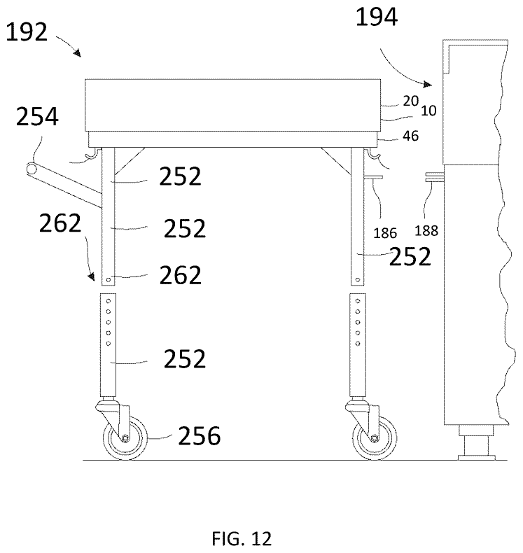

As shown in FIG. 11, the sterilization packaging system 100 may include multiple bases 10 arranged on a rack system 150. The rack system 150 in one example embodiment includes a plurality of shelves 152 for receiving bases 10, or bases 10 having trays 8, which are wrapped in the sterilization wrap 20. Rack system 150 may include sidewalls 154 and a door 156, wherein a plurality of bases 10 and/or bases 10 having trays 8 may be loaded onto the plurality of shelves when the door is in an open position. In one configuration, the shelves 152 are removeable. In another configuration, the shelves are extendable for ease of receiving a base 10. Typically, a plurality of bases 10, each having a wrap 20 encompassing and sealing the base 10, are inserted into the rack system 150 on the shelves 152 after the bases 10 sealed with the wrap 20 have been sterilized in a sterilizer. Thus, a multitude of bases can be stored in the rack system 150 after processing the bases in a sterilizer. The rack system 150, in one exemplary embodiment, includes wheels 158 to allow the rack system 150 holding a plurality of processed bases 10 to be transported into an operating room.

In a configuration, the base 10 includes a locking mechanism to releasably lock the base to a base pedestal, legs, or transfer cart 192. The transfer cart 192 may comprise a frame 252, push handles 254, and wheels 256. If the transfer cart 192 is serving as an operating back room table, the transfer cart 192 may optionally not include push handles 254 or a locking mechanism. The transfer cart 192 can be constructed out of any durable material that is strong enough to maintain its shape and form under significant weight and also able to be sterilized. Ideally, transfer cart 192 is constructed out of a steel or aluminum alloy or a combination thereof. It should be understood that transfer cart 192 is merely an exemplary embodiment of a transfer cart 192.

The transfer cart 192 may further include a sled (not shown) having wheels 260 for sliding along transfer cart 192 guiderails when the sled and base 10 are loaded into a sterilizer. The transfer cart 192 is rigid enough to maintain the weight of the base 10 and sled or other like sterilizing device or apparatus. In one configuration, the base 10 releaseably locks to the sled via a locking mechanism 262. The locking mechanism 262 provides a means to removeably affix the base 10 to the transfer cart 192. Exemplary embodiments of the locking mechanism 262 include any means that would allow a base to be securely affixed a transfer cart 192, for example, a sled of the transfer cart 192, such that the base 10 only moves when transfer cart 192 moves. Additionally, the sled may releasably lock to the transfer cart 192 via a locking mechanism 262, which provides means to removeably affix the sled to the transfer cart 192. Exemplary embodiments of the locking mechanism 262 include any means that would allow a sled to be securely affixed to the transfer cart 192 such that the sled only moves when transfer cart 192 moves. The transfer cart 192 may further include a locking mechanism to releasably lock the transfer cart 192 to the sterilizer. Thus, the transfer cart 192 can be locked relative to the sterilizer when the sled and/or base 10 is loaded into the sterilizer to prevent unwanted movement of the transfer cart 192 during the weight transfer of the load from the transfer cart 192 to the sterilizer. Exemplary embodiments of the locking mechanisms include clamps, laches, slots, bolts, screws, and the like. In another configuration, the base 10 may be fixedly secured to the transfer cart 192. For example, in one configuration, the base 10 is integral and permanently affixed to the transfer cart 192.

Push handles 254 of transfer cart 192 provide the means for a user to more easily push and pull transfer cart 192. Push handles 254 may be provided on opposite sides of transfer cart 192. Each of the push handles 254 span the width of transfer cart 192. It should be appreciated, that embodiments of push handles 254 include push handles 254 being located on all sides or only on one side of transfer cart 192 along with different configurations.

One advantage of having the base 10, such as an operating room back table, loaded directly onto the transfer cart 192 and then into a sterilizer is that once the sterilization cycle is complete, the base 10 (table) can be loaded back onto the transfer cart 192 and transferred directly to the operating room for use again. The instruments or other object, having already been organized on the tray before sterilization, are ready for use without further processing or organizing. In an alternative configuration, the base 10 and transfer cart 192 are loaded together into a ground-loading sterilizer. In this configuration, the transfer cart 192, the base 10, and the instruments contained thereon are sterilized in the ground-loading sterilizer. Thus, the base 10 is not separated from the transfer cart 192 during sterilization or during transportation.

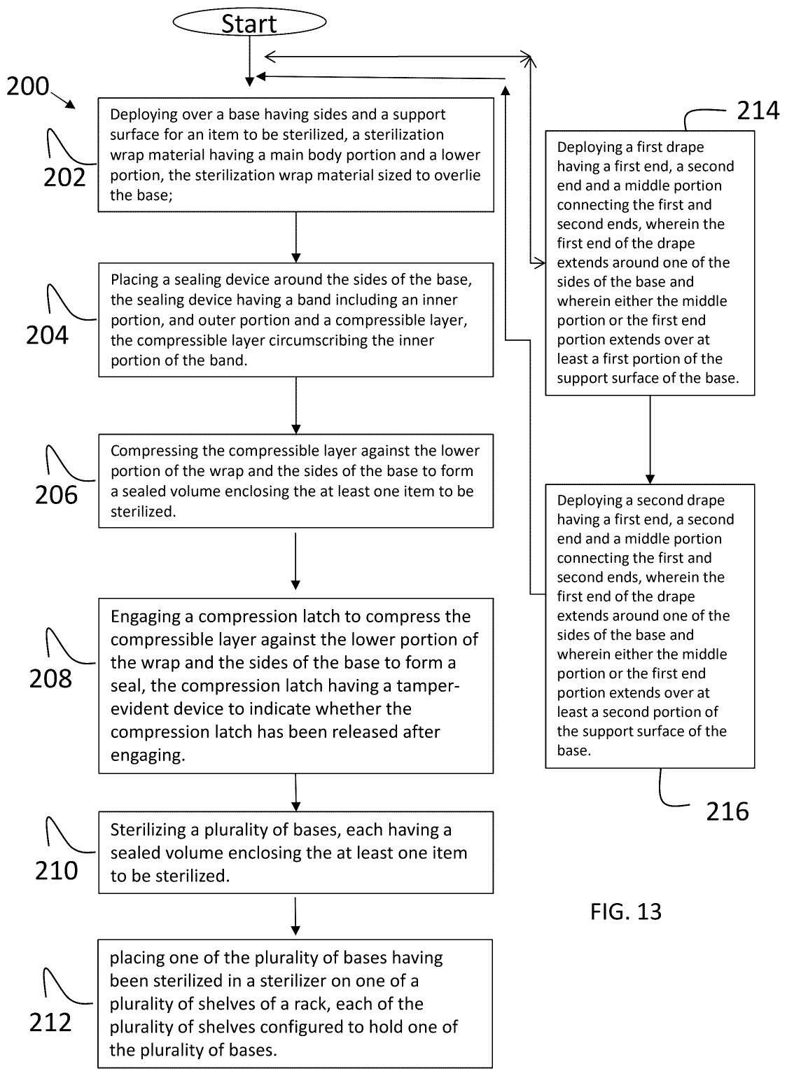

The following steps may be followed with sterilizing items in a sterilizer as shown in FIG. 13. First, according to step 200, a sterilization wrap 20 is deployed over a base 10. In one configuration, the base 10 has a plurality of instruments to be sterilized on a support surface. The sterilization wrap 20 in one configuration includes a main body portion 22 and a lower portion 24 and the sterilization wrap 20 is sized to overlie the base. Optionally, in one configuration, according to step 214, before the sterilization wrap material is deployed, a drape 110 having a first end 112, a second end 114, and a middle portion connecting the first and second ends, is deployed, wherein the first end 112 of the drape 110 extends around one of the sides of the base 10 and wherein either the middle portion or the first end portion 112 extends over at least a first portion of the support surface of the base 10. In this configuration, the base can be base 10 or a tray 8. Further, according to step 216, a drape 130 having a first end 132, a second end 134 and a middle portion connecting the first and second ends 132, 134, wherein the first end 132 of the drape 130 extends around one of the sides of the base and wherein either the middle portion or the first end portion 132 extends over at least a second portion of the support surface of the base.

Next, according to step 204, a sealing device 30 is placed around the sides of the base 10, the sealing device 30 having a band 34 including an inner portion 36, and outer portion 38 and a compressible layer 32, the compressible layer 32 circumscribing the inner portion 36 of the band 34. The compressible layer 32 is compressed against the lower portion 24 of the wrap 20 and the sides of the base 10 to form a sealed volume enclosing the at least one item to be sterilized according to step 206.

A compression latch 28 may be engaged as provided in step 208 to compress the compressible layer against the lower portion 24 of the wrap 20 and the sides of the base 10 to form a seal, the compression latch 28 having a tamper-evident device to indicate whether the compression latch 28 has been released after engaging. As provided in step 210, a plurality of bases 10, each having a sealed volume enclosing the at least one item to be sterilized is sterilized. After sterilization, the plurality of bases is optionally placed on one of a plurality of shelves of a rack 150, each of the plurality of shelves 152 configured to hold one of the plurality of bases according to step 212. As an alternative to this step, a seal may be formed between the wrap 20 and the base 10 using other methods disclosed herein, including but not limited to, adhering the wrap 20 to the base 10.

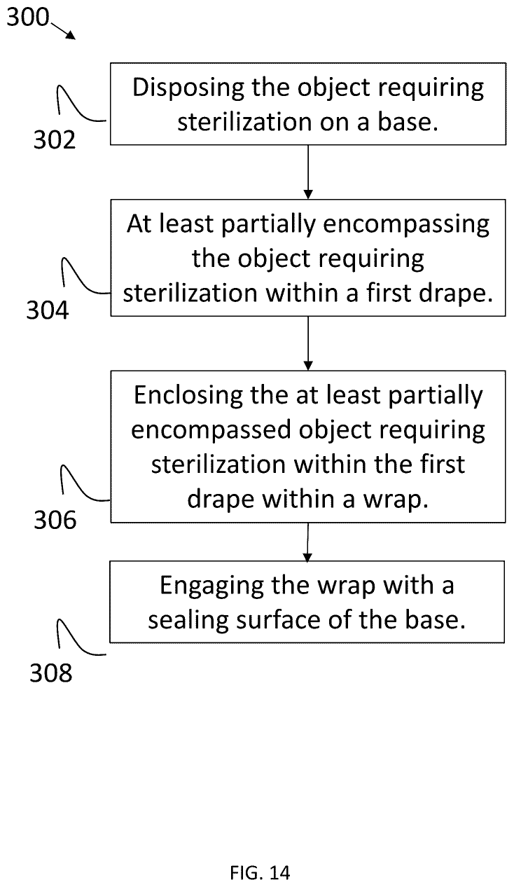

In another exemplary embodiment 300, the following steps may be followed with sterilizing items in a sterilizer as shown in FIG. 14. First, according to step 302, objects requiring sterilization are disposed on a base 10. Next, optionally, the object requiring sterilization is at least partially encompassed within a drape 110 or 130 according to step 304. According to step 306, the at least partially encompassed object requiring sterilization is enclosed within the drape 110 or 130 within a wrap 20. Finally, the wrap is engaged with a sealing surface of the base 10 according to step 308. The sealing surface, in one configuration, is a continuous seal. The sealing surface, in a configuration, is additionally or alternatively a perimetric seal. In one exemplary embodiment, the object is substantially encompassed with the drape 110 or 130. In another exemplary embodiment, the object is substantially encompassed with both the drape 110 and the drape 130. In one exemplary embodiment, the step 306 of enclosing the at least partially encompassed object requiring sterilization within the first drape within a wrap may further include forming at least one corner of the wrap 20, the at least one foldable corner having an inner pleat (for example, 98a, 98b) and a panel (for example, 98c), and folding the corner proximate a side of the wrap 20 to provide continuous wrap material at the corner (as shown in FIG. 5B).

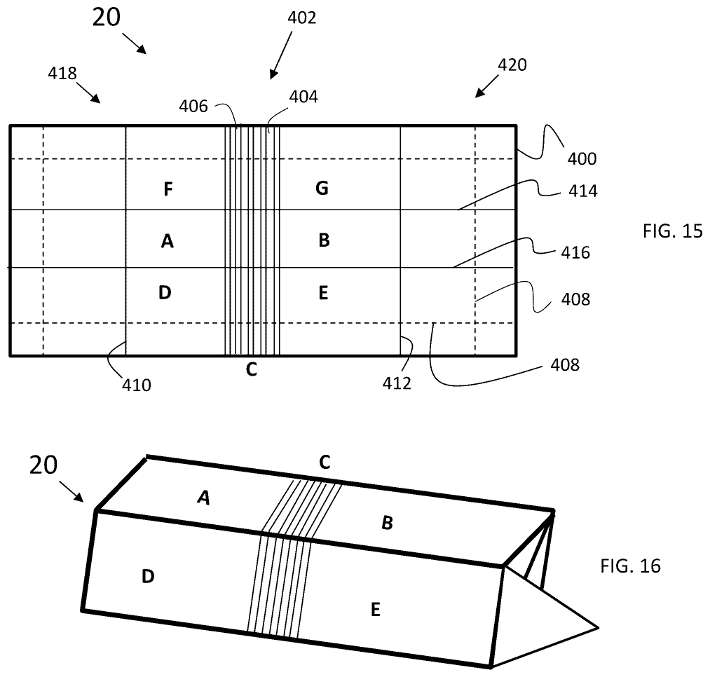

As shown in FIGS. 15 and 16, the sterilization wrap 20 in one exemplary embodiment is expandable. For example, as shown in FIG. 15, the sterilization wrap 20 can comprise a one-piece sheet member 400 having a first portion of plurality of accordion folds 402 which include a series of spaced and substantially parallel plates 404 with alternating raised and recessed folds 406 forming a corrugation pattern of accordion pleats traversing the sterilization wrap material (Area C), wherein the one-piece sheet 400 is oversized to overlie the base 10 having at least one item to be sterilized. In one exemplary embodiment, the one-piece sheet member 400 is free of additional fold lines, wherein the one-piece sheet member 400 is sized to be wrapped around the base 10 to completely cover the base 10. In another exemplary embodiment, the sheet member 400 may include longitudinal fold lines 410, 412, and lateral fold lines 414, 416. Longitudinal fold line 410, lateral fold lines 414, 416, and the first of the accordion folds 402 form top panel A while longitudinal fold line 412, lateral fold lines 414, 416 and the last of the accordion folds 402 form top panel B. Forming side panel D are longitudinal fold line 410, lateral fold line 416, and the first of the accordion folds 402. Forming side panel E are longitudinal fold line 412, lateral fold line 412, and the last of accordion folds 402. Similarly, side panel F is formed by longitudinal fold line 410, lateral fold line 416, and the first of the accordion folds 402. Side panel G is formed by longitudinal fold line 412, lateral fold line 414, and the last of the accordion folds 402. The expandable portion, for example, accordion folds 402 are configured to be moveable between a contracted state to an expanded state. In one example embodiment, this enables the sheet member 400 to be used on various sized bases 10. Alternatively, or additionally, having an expandable wrap 20 enables the wrap 20 to be removed from the base 10 without contaminating the sterilized contents on the base 10. That is, expanding the wrap 20 away from the sterilized surfaces after sterilization when removing the wrap 20 from the base 10 reduces the likelihood that the outside of the sterilization wrap 20 will contact any of the sterilize surfaces encompassed within the wrap 20. In one example embodiment, the top panels and side panels are unseparable from each other. In another configuration, the top and side panels A, D, F are contiguous with each other and form a first section 418 of the sterilization wrap 20 while the top and side panels B, E, G are contiguous with each other and form a second section 420 of the sterilization wrap 20 that is separable from the first section 418. In one example embodiment, the first section 418 of the sterilization wrap 20 and the second section 420 of the sterilization wrap 20 each comprise an accordion fold 402 portion. In another example embodiment, only one of the first section 418 and second section 420 include an accordion fold section 402.

An inner perimeter 408 of the one-piece sheet member 400 may be secured to the base 10. In one exemplary embodiment, the securement device 30 provides a sealed interface between the securement device 30 and the one-piece sheet member 400 and also between the base 10 and the one-piece sheet member.

In one example embodiment, the one-piece sheet member 400 is folded along the longitudinal fold lines 410, 412 and the lateral fold lines 414, 416 to form folds along each of the longitudinal fold lines and the lateral fold line, respectively. Each of the fold lines 410, 412, 414, 416, in one example embodiment, are then reinforced to form a pre-shaped bag. It should be appreciated that the wrap 20 shown in FIGS. 15 and 16 may be used with the sterilization packaging system shown in FIGS. 1, 2, 7, 8, 9, and 10 and may include the additional features described herein. By way of non-limiting example, the securement device 30 described in FIGS. 1-3 may be used with the wrap 20 shown in FIGS. 15 and 16.

The present invention contemplates that many changes and modifications may be made. Therefore, while the presently-preferred form of the apparatus and method has been shown and described, and several modifications and alternatives discussed, persons skilled in the art will readily appreciate that various additional changes and modifications may be made without departing from the scope of the invention, as defined and differentiated by the following claims.

1. A sterilization packaging system for an object requiring sterilization in a sterilizer, the sterilization packaging system comprising:
a base having a support surface and a plurality of sides, the support surface configured to support an object requiring sterilization;
a wrap sized to overlie the sides of the base and the object requiring sterilization, the wrap having a main body portion and a lower portion;
a sealing interface between the lower portion of the wrap and the base, the sealing interface configured to form a continuous seal between the lower portion of the wrap and a first sealing surface of the base; and
a first drape moveable between an unfolded position and a folded position, wherein the first drape is sized to overlie at least a first portion of a support surface of the base in the folded position.

2. The sterilization packaging system of claim 1, further comprising a second drape moveable between an unfolded position and a folded position, wherein the second drape is sized to overlie at least a second portion of a support surface of the base in the folded position.

3. The sterilization packaging system of claim 1, wherein the first drape further comprises a first end, a second end and a middle portion connecting the first and second ends, wherein, in the folded position, the first end of the first drape overlaps the second end of the first drape over the support surface.

4. The sterilization packaging system of claim 2, wherein the second drape further comprises a first end, a second end and a middle portion connecting the first and second ends, wherein, in the folded position, the first end of the second drape overlaps the second end of the second drape over the support surface.

5. The sterilization packaging system of claim 1, wherein the first sealing surface is a perimeter of the base.

6. The sterilization packaging system of claim 2, wherein at least one of the first drape and second drape is between the sealing interface of the lower portion of the wrap and the base.

7. The sterilization packaging system of claim 1, wherein the base is a tray.

8. The sterilization packaging system of claim 1, wherein the base is a table having the plurality of sides, and wherein the plurality of sides of the table form a dependent skirt.

9. The sterilization packaging system of claim 8, further comprising a clamp configured to form a seal between the dependent skirt of the base and the lower portion of the wrap.

10. The sterilization packaging system of claim 9, wherein the clamp is a band having an inner portion, an outer portion, and a compressible layer, the compressible layer circumscribing the inner portion of the band, wherein the band compresses the compressible layer against the wrap and the dependent skirt of the base to form a seal.

11. The sterilization packaging system of claim 10, wherein the band further comprises a compression latch moveable between an open position to install and release the band and a closed position to form a compression seal, the compression latch having a tamper-evident device to indicate whether the compression latch has been moved from the closed to the open position to release the band.

12. The sterilization packaging system of claim 1, wherein the lower portion of the wrap includes a tear away portion comprising a tear line and a tear tab, and wherein the main body portion is detachably connected to the lower potion along the tear line.

13. The sterilization packaging system of claim 12, wherein the tear line comprises a scored line along the perimeter of the lower portion of the wrap.

14. The sterilization packaging system of claim 1, further comprising a non-expansive device to contain the wrap during the sterilization process, and wherein the non-expansive device is a strap that secures to at least two of plurality of sides of the base.

15. The sterilization packaging system of claim 1, wherein the base further comprises wrap supports extending from the base, the wrap supports being at least one of collapsible and removeable.

16. The sterilization packaging system of claim 2, further comprising:
a plurality of bases, each base having the wrap, the first drape, the second drape, and a continuous seal between the lower portion of the wrap and the first sealing surface; and
a rack having a plurality of shelves, each of the plurality of shelves configured to hold one of the plurality of bases having the wrap after sterilization in a sterilizer.

17. The sterilization packaging system of claim 2, wherein the wrap and base define an interior area and wherein at least one of the first drape and second drape form a secondary seal within the interior area.

18. A sterilization packaging system for an object requiring sterilization in a sterilizer, the sterilization packaging system comprising:
a base having a support surface and a continuous sealing surface, the support surface configured to support the object requiring sterilization;
a first drape sized to overlay the base and at least a first portion of the object requiring sterilization; and
a wrap sized to engage the continuous sealing surface to encompass the first drape and the object requiring sterilization.

19. The sterilization packaging system of claim 18, further comprising a second drape sized to overlie at least a second portion of the object requiring sterilization.

20. The sterilization packaging system of claim 18, further comprising a retainer configured to retain the wrap relative to the continuous sealing surface.

21. The sterilization packaging system of claim 20, wherein the retainer is further configured to engage the wrap with the continuous sealing surface.

22. The sterilization packaging system of claim 20, wherein the retainer further comprises a compressible layer and a band, the band moveable between a release configuration and a sealing configuration.

23. The sterilization packaging system of claim 20, wherein the retainer includes a tamper evident element.

24. The sterilization packaging system of claim 18, wherein the continuous sealing surface defines a periphery of the base.

25. The sterilization packaging system of claim 20, further comprising:
a plurality of bases, each base having the wrap, the first drape, and the retainer retaining the wrap relative to the continuous sealing surface; and
a rack having a plurality of shelves, each of the plurality of shelves configured to hold one of the plurality of bases having the wrap after sterilization in a sterilizer.

26. A method of preparing an object requiring sterilization in a sterilizer, the method comprising:
disposing the object requiring sterilization on a base;
at least partially encompassing the object requiring sterilization within a first drape;
enclosing the at least partially encompassed object requiring sterilization within the first drape within a wrap; and
engaging the wrap with a continuous sealing surface of the base.

27. The method of claim 26, wherein engaging the wrap with the continuous sealing surface of the base includes disposing a band in an engaging configuration to retain a portion of the wrap between the band and the continuous sealing surface of the base.

28. The method of claim 26, wherein a portion of the band includes a compressible cellular layer.

29. The method of claim 26, further comprising at least partially encompassing a second portion the object requiring sterilization within a second drape.

30. The method of claim 26, wherein the step of enclosing the at least partially encompassed object requiring sterilization within the first drape within a wrap further includes forming at least one corner of the wrap, the at least one foldable corner having an inner pleat and a panel, and, folding the corner proximate a side of the wrap to provide continuous wrap material at the corner.
